(19) United States
(12) Patent Application Publication
(10) Pub. No.: US 2017/0232127 A1
(43) Pub. Date: Aug. 17, 2017

(54) GARBAGE DEODORIZING SYSTEM

(71) Applicant: Ozone Generation, LLC, Jacksonville, FL (US)

(72) Inventor: Audrius Bakanas, Jacksonville, FL (US)

(21) Appl. No.: 15/427,901

(22) Filed: Feb. 8, 2017

(30) Foreign Application Priority Data
Feb. 8, 2016 (GB) 1602191.7

(51) Int. Cl. A61L 9/12; C01B 13/02; B65F 1/14; A61L 9/04

(52) U.S. Cl. CPC A61L 9/12; A61L 9/046; C01B 13/02; B65F 1/14; A61L 2209/212

(57) ABSTRACT

A system for sanitizing and deodorizing garbage chutes using methane sensors in the trash chute that includes an ozone generating unit at the bottom of the trash chute. The system can include an ozone sensor as a safety device to ensure that ozone levels do not surpass a predetermined amount. The present invention works with a controller that controls the ozone generator based on a schedule program for a predetermined time of day. The present invention in an embodiment can include a plurality of additional sensors such as airflow detectors, occupancy detectors and the like.

### OTHER RELATED APPLICATIONS.

The present application claims priority to an application filed in the United Kingdom with application Serial No. 1602191.7 filed on Feb. 8, 2016.

### BACKGROUND OF THE INVENTION

1. Field of the Invention

The present invention concerns a garbage deodorizing system and, in particular, a system and installation for sanitizing garbage stores.

Modern society produces vast quantities of waste. At the same time, public services are becoming increasingly stretched, leading to less frequent garbage collection.

Consequently, the safe storage of garbage between collections is becoming a more challenging issue, particularly in areas of dense population, such as in cities. For example, many multi-story buildings have communal garbage stores where huge amounts of refuse may be collected from throughout the building. Such garbage stores are typically provided as a dedicated room for housing large garbage bins, with the stores normally located in the building's ground or basement level. Garbage accumulated in these garbage stores commonly includes food and other organic waste which rots, providing a breeding ground for pests as well as unwanted pollutants, such as bacteria, protozoa, mold and other fungi, as well as other organic pollutants. Moreover, concentrations of fungi and bacteria not only accumulate in the garbage store, but can also become airborne. Garbage stores therefore present a major health hazard, and a poorly maintained garbage store may quickly become infested with verminous pests and plagued by foul odors.

The above issues are exacerbated in buildings which have garbage collection chutes or tubes for tunneling garbage from different floors of the building to the garbage store. In such instances, airborne pollutants can permeate through the network of garbage chutes and get transported around the building via air circulation. In such cases, garbage store maintenance becomes even more important.

2. Description of the Related Art.

Traditional methods for disinfecting and deodorizing a garbage collection system involves a maintenance program of regularly washing the garbage store and any associated collection chutes with high-pressured water and chemicals.

However, this is an expensive, labor intensive process and, as the flow of trash is continual, a garbage store can get filthy again very quickly. Furthermore, the water and chemicals used for cleaning does not always penetrate all the small cracks and crevices of a garbage collection system where the sources of bad smells may reside. To mitigate the issue of bad smells, particularly between cleaning cycles, an independent system to control bad smells is often utilized. One such system is a time-release pump that sprays the garbage chutes and/or receptacles with a scented chemical odor. However, whilst such a system may help to mask bad smells, such a method does not address the source of foul odors. Moreover, chemical sanitizing agents often do not easily decompose, leaving toxic chemical residues that are themselves hazardous to human health.

Traditional methods for disinfecting and deodorizing a garbage collection system involves a maintenance program of regularly washing the garbage store and any associated collection chutes with high-pressured water and chemicals.

However, this is an expensive, labor intensive process and, as the flow of trash is continual, a garbage store can get filthy again very quickly. Furthermore, the water and chemicals used for cleaning does not always penetrate all the small cracks and crevices of a garbage collection system where the sources of bad smells may reside. To mitigate the issue of bad smells, particularly between cleaning cycles, an independent system to control bad smells is often utilized. One such system is a time-release pump that sprays the garbage chutes and/or receptacles with a scented chemical odor. However, whilst such a system may help to mask bad smells, such a method does not address the source of foul odors. Moreover, chemical sanitizing agents often do not easily decompose, leaving toxic chemical residues that are themselves hazardous to human health.

More recently, automated garbage bin store disinfection systems have been proposed that neutralize and control both surface and airborne mold, bacteria and other harmful microorganisms. One such system is disclosed in US 2015/0157753 which focuses on preventing garbage smells from reaching the occupied floors of a building through inlets to the garbage chute on each occupied floor. With US 2015/0157753, a methane sensor is located at the top of a garbage chute and, in response to elevated methane levels, an ozone generator located within the garbage store is controlled to generate ozone. The ozone gas is an effective disinfectant and is able to efficiently disinfect a large air volume to prevent a build-up of bio-films, bacteria, and fungi within the garbage store and its connecting chutes.

As such, the use of an ozone generator in the garbage store acts to eliminate bad smells and the spread of health problems such as allergies, rashes, colds, viruses and legionnaires disease among others. Ozone is also known to neutralize mold build up, and deters vermin. Consequently, the system disclosed in US 2015/0157753 can significantly improve sanitary conditions. However, the conventional ozone garbage deodorizing system disclosed in US 2015/0157753 suffers from a number of shortcomings.

### SUMMARY OF THE INVENTION

Firstly, the system of US 2015/0157753 merely generates ozone in response to the detection of elevated methane levels at the top of the garbage chute. As such, US 2015/0157753 is only suitable for garbage collection arrangements that include a garbage chute. The system of US 2015/0157753 also does not directly address other pollutants which may be present in the garbage store itself.

For example, there may be periods where less food waste is deposited or a colder ambient temperature means that the rate of food rotting is lower. Such situations would lead to a corresponding reduction in the level of methane gas generated, which may cause the system of US 2015/0157753 to be less effective at addressing residual biofilms, mold, or other pests that may still be present in the garbage store. Furthermore, whilst the system taught by US 2015/0157753 is effective for preventing garbage smells at the inlets to the garbage chute, such smells may be replaced by the smell of ozone gas, which itself has a notably pungent smell. Moreover, ozone gas is a hazardous substance and high concentrations in the garbage store can make it unpleasant for persons who are either located close 35 to the garbage store or those who need to physically access the garbage store, for instance to empty the garbage bins. Prolonged exposure to elevated levels of ozone can even lead to headaches, eye and respiratory irritation and potentially more long term negative effects, including lung damage. To 5 regulate the quantities of ozone produced, the system of US 2015/0157753 does propose embodiments in which an ozone sensor is used to monitor ozone levels in the garbage chute and store, and then shut off the ozone generator if concentrations exceed a predetermined level. However, whilst this type of simple safety cut off mechanism can avoid dangerously high ozone levels being generated, it does not take into account other factors which may have a bearing on what level of ozone concentration might be acceptable.

The present invention therefore seeks to address some of the above issues with conventional garbage deodorizing systems. According to a first aspect of the present invention there is provided a garbage deodorizing system for installation into a garbage store, comprising: an ozone generator for generating ozone in the garbage store; an ozone sensor for detecting ozone concentration in the garbage store; and a controller for controlling the ozone generator to generate ozone based on the detected ozone concentration and a determined time of day.

In this way, embodiments of the present invention are able to control the amount of ozone in a garbage store's 30 ambient air throughout the day to strike an optimized balance between effective sanitation and a safe environment for users. For example, higher ozone concentrations provide an improved sanitizing effect. However, if such high concentrations of ozone are maintained at times of the day when the garbage store or associated garbage chutes are accessed frequently, users are placed at much higher risk of exposure to unpleasant or detrimental levels of ozone. Embodiments of the present invention are therefore able to address this by, for example, generating higher concentrations at night or during the middle of the day when users do not generally deposit garbage in the garbage store. This allows the effectiveness of the ozone generated in combatting microorganisms or pests present in garbage collection system to be maximized, whilst nevertheless ensuring that any relevant regulatory limits are not exceeded. Then, at times of the day when the garbage store is more frequently accessed, ozone levels can be allowed to drop to a lower level to avoid any detriment to users.

Preferably, the controller comprises a timer for determining the time of day. This offers a simple mechanism for monitoring a 24-hour schedule, although other methods are possible. For instance, an outdoor daylight and/or temperature sensor may be provided to differentiate between daytime and nighttime.

Preferably, the controller controls the ozone generator based on a schedule program and the determined time of day. This allows a timing schedule for operation of the ozone generator to be easily defined.

Preferably, the timing program further accounts for one or more of: the time of year, working days, and holidays. In this way, the amount of ozone generated throughout the day can be varied to take into account seasonal conditions or occupancy patterns. For example, colder temperatures over winter may mean garbage odors are less of an issue compared to the summer months. Equally, a garbage system may be accessed much less frequently during the daytime of a weekday, as opposed to weekends or holidays.

Preferably, the controller stops the ozone generator from generating ozone if the ozone detector detects ozone exceeding a designated stop concentration level. This allows embodiments to avoid generating excessive ozone. This may be used for instance to avoid exceeding regulatory limits or a level beyond which ozone becomes detectable to a building's residents.

Preferably, the designated stop concentration level is set at different levels for different times of day. In this way, at times of the day where high ozone concentrations are intended, the designated stop concentration level may be based on the allowed regulatory limits, whereas at times of the day where low ozone concentrations are intended, a lower level may be set which avoids detection by a building's residents.

Preferably, the ozone sensor is provided in a housing separate to the ozone generator. This thereby allows the ozone sensor to be mounted away from the source of the ozone, thereby allowing ozone to mix with the ambient air before detection. This provides for a more accurate measurement of ozone concentration within the garbage store.

Preferably, the garbage deodorizing system further comprises an occupancy detector for detecting the occupancy of the garbage store by a person, and wherein the controller stops the ozone generator from generating ozone if the occupancy detector detects a person in the garbage store. This provides an additional safety mechanism since, although ozone still lingers in ambient air within the garbage store, it is no longer being actively generated and therefore will dissipate. This allows a person to safely use the premises and avoids prolonged exposure to very high ozone levels, which could be detrimental to their health.

Preferably, the occupancy detector is a motion detector. As such, movement of a person may be used to determine their presence. Various motion detector technology may be used, such as infrared, microwave, ultrasonic, tomographic sensors. Other types of occupancy detector may also be used, such as, for example, door sensors which detect when a door to the garbage store has been opened. Furthermore, combinations of sensors may be used in the occupancy detector to provide more accurate detection.

Preferably, the garbage deodorizing system further comprises an airflow detector for detecting airflow vented from the garbage store, and wherein the controller stops the ozone generator from generating ozone if the airflow detector detects that the rate of airflow drops below a threshold. In this way, a clog in the venting arrangement used by the garbage systems may be quickly detected to avoid a rapid build up of ozone gas.

Preferably, the airflow detector comprises mounts for installation at a top of a garbage chute. In this way, any clogging of the garbage chute may be quickly detected.

Preferably, the garbage deodorizing system further comprises a second ozone detector for detecting ozone in a garbage chute, and wherein the controller stops the ozone generator from generating ozone if the second ozone detector detects ozone exceeding a designated second stop concentration level. In this way, a second fail safe is provided to avoid excessive ozone levels reaching the occupied floors of a building though the garbage chute inlets.

Preferably, the designated second stop concentration level is set at different levels for different times of day. In this way, the system is able to capitalize on times of the day where the inlets to the garbage chute are unlikely to be opened, thereby permitting higher ozone concentrations at these times.

Preferably, the designated second ozone detector comprises mounts for installation at a top of a garbage chute.

Preferably, the garbage deodorizing system further comprises a central controller for receiving feedback data from the controller. In this way, the central controller can allow for remote supervision of the garbage deodorizing system.

Preferably, the central controller comprises an input for receiving other feedback data from other garbage stores. In this way, multiple garbage stores may be monitored and compared.

Preferably, the feedback data is transmitted from the controller to the central controller via one of a cable connection, a GSM module, or an internet connection.

According to a second aspect of the present invention, there is provided a garbage collection installation, comprising a garbage store for storing garbage; an ozone generator for generating ozone in the garbage store and being mounted to a wall of the garbage store; an ozone sensor for detecting ozone concentration in the garbage store and being housed in a separate unit to the ozone generator and mounted to an opposing wall of the garbage store; and a controller for controlling the ozone generator to generate ozone based on the detected ozone concentration and a determined time of day.

### BRIEF DESCRIPTION OF THE DRAWINGS

Illustrative embodiments of the present invention will now be described with reference to the accompanying drawings in which:

FIG. 1 shows a garbage deodorizing system according to a first embodiment of the invention; and

FIG. 2 shows a garbage deodorizing system according to a second embodiment of the invention.

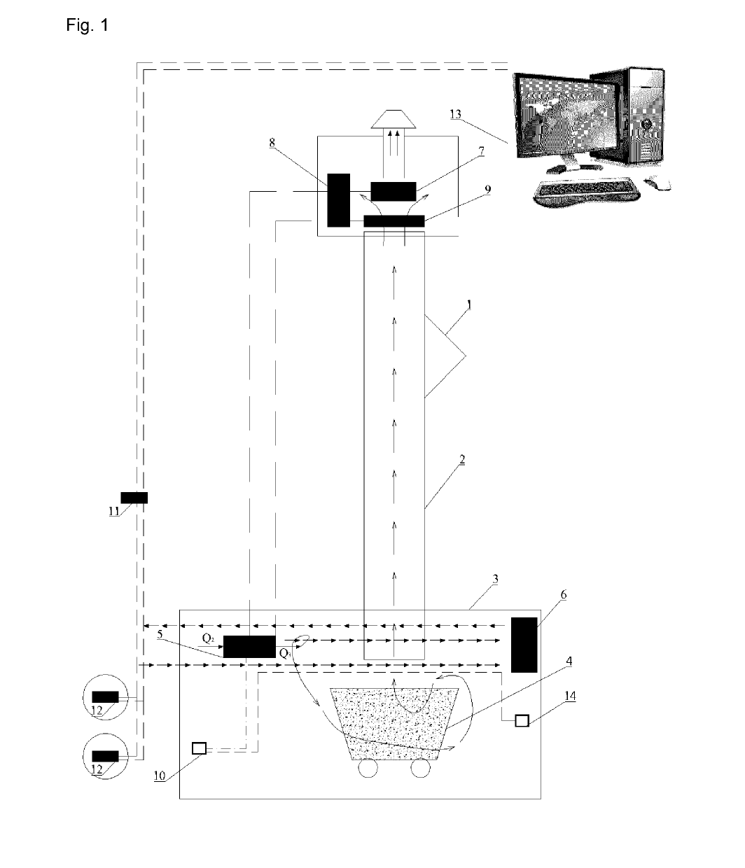

FIG. 1 shows a schematic diagram of a garbage deodorizing system according to a first embodiment of the invention. In this embodiment, the garbage deodorizing system is installed into a garbage store 3 which is connected to a garbage chute 2, which can be accessed through intake door 1. The top of the garbage chute 2 has a venting chimney. In use, a user of the garbage collection system disposes of rubbish through intake door 1, from where it falls down chute 2 into a garbage bin 4 located in the garbage store 3.

The garbage deodorizing system comprises an ozone generator 5 which emits ozone into the garbage store 3 under the control of control panel 10. The ozone outlet of the ozone generator is preferably configured to direct a stream of ozone gas into the garbage bin 4. The control panel 10 includes a timer for monitoring the time of day and a timer schedule program for controlling the ozone generator's activity based on the determined time of day. In this embodiment, the control panel 10 comprises a touch screen for inputting control options and programming the timer schedule program.

A central controller 13 is also provided in this embodiment in the form of a computer for receiving control information from control panel 10, as well as other control panels 12 associated with other garbage deodorizing systems located in other garbage stores, for example, in the same building complex. Central controller 13 is able to monitor the received control information and centrally adjust settings by providing feedback instructions to control panels 10 and 12. In this embodiment, data received and transmitted between central controller 13 and the control panels 10 and 12 is directed through data accumulation unit 11.

Within the garbage store 3 is located ozone sensor 6 for sensing the ozone concentration within the store itself, and motion sensor 14 for detecting if the garbage store is occupied by a person. Both sensors transmit sensed data to control panel 10, in this embodiment via wired connections, although a wireless connection may also be used. The ozone sensor 6 is provided in a housing separate to the ozone generator 5 and is fitted to a wall of the garbage store 3 at location away from the ozone generator, and preferably on an opposing wall of the garbage store to provide the maximum separation between the units. This is to allow any generated ozone to mix with the ambient air before reaching the ozone sensor 6. This thereby helps to provide a more accurate reading of ozone concentration at the ozone sensor.

Motion sensor 14 is mounted to a wall of the garbage store and is configured to minimize the risk of being inadvertently triggered by other movements caused by, for example, falling garbage or moving rodents. In this embodiment, this is achieved by directing the sensor of motion detector 14 toward a location where only the movement caused by a person entering the garbage store 3 is detected. However, other techniques may be used, such as using passive infrared sensor configured to ignore heat signatures smaller than that of a person. In such an arrangement, occupancy by a person may thereby be detected, whereas radiation emitted from any rodents will be ignored.

Air flow meter 9 is located at the top of garbage chute 2 at the junction with the venting chimney, and is used to measure the airflow vented from the garbage chute 2. A further ozone sensor 7 is provided for detecting the ozone levels in the air leaving air flow meter 9. A control relay 8 is provided for feeding the sensed data back to control panel 10.

The garbage deodorizing system of FIG. 1 monitors and controls the amount of ozone in a garbage store's ambient air and strikes a balance between effective sanitation and a safe environment for users of the garbage store. In this connection, in use, the control panel 10 uses its timer and the programmed timer schedule to control the operation of ozone generator 5 to vary the amount of ozone generated at different times and may be configured to account for the size of a garbage collection system, the ambient air temperature, and humidity and frequency of garbage disposal in order to maximize the effectiveness of the ozone generated in combatting microorganisms present in garbage collection system. In this respect, the effectiveness of the ozone as a sanitizer depends on several factors, including the amount of ozone applied, the residual ozone in the medium, and various other environmental factors such as medium pH, temperature, relative humidity, and the amount of organic matter in the garbage. For example, the control panel 10 uses the timer to regulate the times that the ozone generator is active in order to optimize effectiveness. For example, the control panel 10 may activate ozone generator 5 at night, when the garbage store 3 is not normally used, and then turn off the generator 5 before the day starts. Alternatively, the ozone generator may remain switched on, but the rate of ozone emitted can be adjusted to a lower level. Moreover, the control panel 10 can be programmed to account for time of year, work days and holidays. The control panel 10 therefore offers users of the garbage store the flexibility to set an ozone limit based on many environmental factors, personal preferences and the dirtiness of the garbage store, while avoiding exceeding regulatory ozone limits.

In this connection, ozone sensors 6 and 7 are used to detect when ozone levels have exceeded designated limits within the garbage store 3 and garbage chute 2, respectively, and in response to such a detection, control panel 10 turns ozone generator 5 off to prevent a further increase in ozone concentration. The designated limits for each location may be different since higher ozone concentrations may be tolerated in the garbage store 3, as compared to the garbage chute 2, where very high ozone concentrations could leak out through door 1 to the occupied floors of the building. Furthermore, the designated limits may also be varied for different times of day based on the timing schedule program. For example, overnight, a high ozone concentration limit may set based on the maximum allowed regulatory ozone limit. Conversely, during the day time when the garbage store is more likely to be accessed, a lower ozone concentration limit may be set to avoid any perceptible detection of ozone by a building's residents. Once the ozone levels detected by ozone sensors 6 and 7 have dropped back below a preset acceptable level, control panel 10 may then prompt ozone generator 5 to resume generation of more ozone. In this way, excessively high concentrations of ozone for a given situation can be avoided.

Motion sensor 14 is used to detect if a person enters the garbage store 3 and, in such an event, causes the control panel 10 to turn off ozone generator 5. Although ozone still lingers in the ambient air within the garbage store 3, it is no longer being actively generated and therefore a person can safely use the premises. Furthermore, as a person would typically enter the garbage store via an access door, its opening would allow ozone to escape. This has the effect of reducing the ozone concentration within garbage store 3, with this occurring at the same time as the detected occupancy prevents new ozone from being generated. As such, ozone levels within the garbage store 3 will drop when a person accesses the garbage store, for example to empty garbage bin 4 or manually clean the room. This thereby avoids exposure to very high ozone levels, which could be detrimental for human health.

In this connection, the rate at which ozone levels drop in the event that the access door to the garbage store is opened may also have a bearing on how high the ozone concentration limit may be set. For example, factors such as the garbage store opening onto the outside of a building or having a large entrance door may mean that ozone in the garbage store is quickly dissipated when the door is opened. Consequently, the control panel 10 may apply a higher ozone concentration limit, allowing greater levels of ozone to be generated within the garbage store for maximizing sanitization. If a person then accesses the store, the ozone level will drop to an acceptable limit sufficiently quickly to avoid harming them. The control panel 10 or central controller 13 may monitor the rate at which ozone is dissipated from the garbage store and adjust the set ozone concentration limits accordingly.

Air flow meter 9 measures the airflow vented from garbage chute 2. However, in the event that chute 2 gets clogged, the airflow through the chute 2 will be reduced or substantially stopped. Consequently, this reduction in normal airflow can be detected by air flow meter 9, which communicates this back to control panel 10. Control panel 10 may then relay this information as an alert to central control 13 and/or turn off the ozone generator 5 to prevent excessive ozone concentrations from building within the garbage store 3.

Data from the control panel timer is collected in data accumulation unit 11, which also collects timer data from control panels 12 in other garbage stores. Data is then transferred from data accumulation unit 11 to central control 13 via, in this embodiment, a wired connection.

Each garbage deodorizing system is able to function as a stand-alone system. However, such a system might not be error-proof and there could be instances of false alarms caused, for example, by electrical failures, user error, poor application engineering, power surges, lightning, and faulty equipment. Therefore, the communications with the central control 13 allow the performance of individual deodorizing systems to be remotely supervised. This arrangement therefore allows for 24-hour monitoring of the garbage deodorizing systems and facilitates a rapid response in case a malfunction occurs somewhere in the network, for example if ozone levels exceed those of regulatory limits.

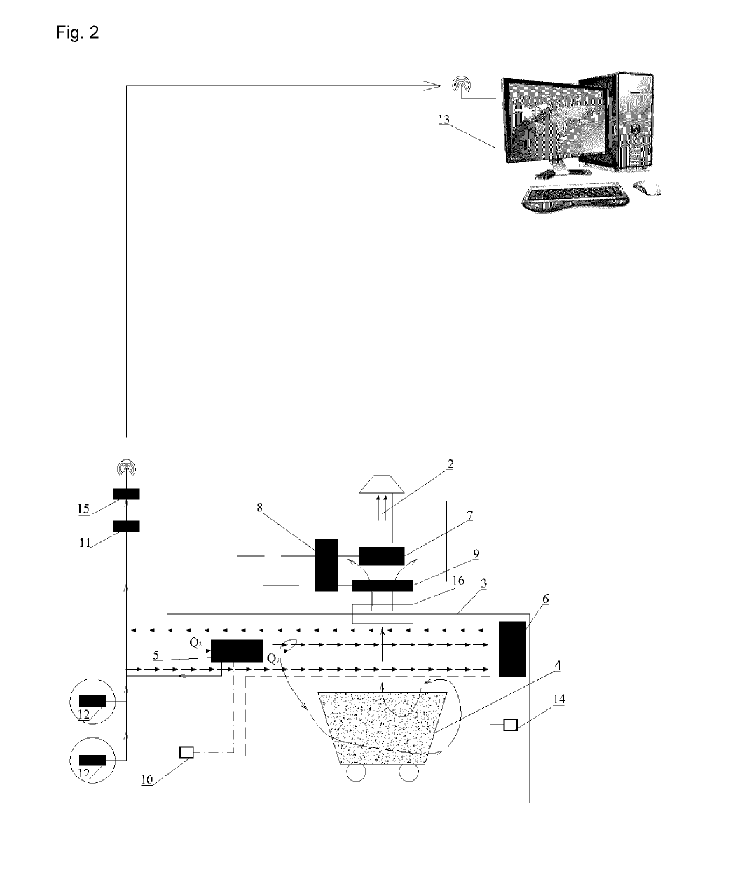

FIG. 2 shows a garbage deodorizing system according to a second embodiment of the invention. This embodiment is similar to the embodiment shown in FIG. 1 and functions in the same way. However, in this embodiment, rather than a dedicated wired connection, transmitter 15 is used to transfer data between data accumulation unit 11 and central controller 13. The transmitter 15 may be, for example, a Wi-fi transmitter, and internet connection terminal, or a GSM cellular network transmitter. Furthermore, in this embodiment, the garbage deodorizing system has been installed into a garbage store 3 which does not have a garbage chute. Instead, the roof of the garbage store 3 is provided with vent 16 and the air flow meter 9 connects the vent 16 with venting chimney 2. The remainder of the components of the garbage deodorizing system shown in FIG. 2, as well as their operation, are the same as those shown in FIG. 1.

### DETAILED DESCRIPTION OF THE EMBODIMENT OF THE INVENTION

As will be understood, embodiments of the present invention allow the level of ozone in the garbage store to be controlled to sanitize the garbage therein, as well as avoid potential detrimental effects to users. Furthermore, the feedback systems ensure that ozone levels do not exceed that mandated by any relevant regulatory agencies.

It will be understood that the embodiment illustrated above show applications of the invention only for the purposes of illustration. In practice the invention may be applied to many different configurations, the detailed embodiments being straightforward for those skilled in the art to implement.

In this connection, for example, the deodorizing system can be altered to incorporate several ozone generators and/or ozone sensors that may be installed or mounted in various locations within the garbage collection system depending on the height of a building and/or layout of a garbage store, to ensure the most effective sanitation.

Furthermore, it will be appreciated that although the present invention functions as a garbage deodorizing system, the use of ozone in the deodorizing technique will have a disinfecting and sanitizing effect on the garbage within the store.

What is claimed is:

1. A garbage deodorizing system for installation into a garbage store, comprising:
a. an ozone generator for generating ozone in the garbage store;
b. an ozone sensor for detecting ozone concentration in the garbage store; and
c. a controller for controlling the ozone generator to generate ozone based on the detected ozone concentration and a determined time of day.

2. A garbage deodorizing system according to claim 1, wherein the controller comprises a timer for determining the time of day.

3. A garbage deodorizing system according to claim 1 or 2, wherein the controller controls the ozone generator based on a schedule program and the determined time of day.

4. A garbage deodorizing system according to claim 3, wherein the timing program further accounts for one or more of: the time of year, working days, and holidays.

5. A garbage deodorizing system according to any preceding claim, wherein the controller stops the ozone generator from generating ozone if the ozone detector detects ozone exceeding a designated stop concentration level.

6. A garbage deodorizing system according to claim 5, wherein the designated stop concentration level is set at different levels for different times of day.

7. A garbage deodorizing system according to any preceding claim, wherein the ozone sensor is provided in a housing separate to the ozone generator.

8. A garbage deodorizing system according to any preceding claim, further comprising an occupancy detector for detecting the occupancy of the garbage store by a person, and wherein the controller stops the ozone generator from generating ozone if the occupancy detector detects a person in the garbage store.

9. A garbage deodorizing system according to any preceding claim, wherein the occupancy detector is a motion detector.

10. A garbage deodorizing system according to any preceding claim, further comprising an airflow detector for detecting airflow vented from the garbage store, and wherein the controller stops the ozone generator from generating ozone if the airflow detector detects that the rate of airflow drops below a threshold.

11. A garbage deodorizing system according to any preceding claim, wherein the airflow detector comprises mounts for installation at a top of a garbage chute.

12. A garbage deodorizing system according to any preceding claim, further comprising a second ozone detector for detecting ozone in a garbage chute, and
wherein the controller stops the ozone generator from generating ozone if the second ozone detector detects ozone exceeding a designated second stop concentration level.

13. A garbage deodorizing system according to claim 12, wherein the designated second stop concentration level is set at different levels for different times of day.

14. A garbage deodorizing system according to claim 12 or 13, wherein the designated second ozone detector comprises mounts for installation at a top of a garbage chute.

15. A garbage deodorizing system according to any proceeding claim, further comprises a central controller for receiving feedback data from the controller.

16. A garbage deodorizing system according to claim 15, wherein the central controller comprises an input for receiving other feedback data from other garbage stores.

17. A garbage deodorizing system according to claim 15 or 16, wherein the feedback data is transmitted from the controller to the central controller via one of a cable connection, a GSM module, or an internet connection.

18. A garbage collection installation, comprising:
a garbage store for storing garbage; an ozone generator for generating ozone in the garbage store and being mounted to a wall of the garbage store; a n ozone sensor for detecting ozone concentration in the garbage store and being housed in a separate unit to the ozone generator and mounted to an opposing wall of the garbage store; and a controller for controlling the ozone generator to generate ozone based on the detected ozone concentration and a determined time of day.

19. A garbage deodorizing system substantially as hereinbefore described with reference to the accompanying drawings.

20. A garbage collection installation substantially as hereinbefore described with reference to the accompanying drawings.
